(19) United States
(12) Patent Application Publication
(10) Pub. No.: US 2017/0199122 A1
(43) Pub. Date: Jul. 13, 2017

(54) METHOD OF DETERMINING A VALUE OF A VARIABLE OF INTEREST OF A SAMPLE HAVING ORGANISMS AND SYSTEM THEREFORE

(71) Applicant: XPERTSEA SOLUTIONS INC., Québec (CA)

(72) Inventors: Valérie ROBITAILLE, Québec (CA); Cody ANDREWS, Québec (CA)

(21) Appl. No.: 15/320,874

(22) PCT Filed: Jul. 7, 2015
(86) PCT No.: PCT/CA2015/050629

§ 371 Date: Dec. 21, 2016

Related U.S. Application Data
(60) Provisional application No. 62/021,556, filed on Jul. 7, 2014.

(51) Int. Cl. G01N 21/51; G06T 7/70; H04N 7/18; G01N 33/18; G01N 15/06

(52) U.S. Cl. CPC G01N 21/51; G01N 33/18; G01N 15/06; H04N 7/183; G06T 7/70; G01N 2015/0693; G01N 2015/0065

(57) ABSTRACT

The method of determining a variable of interest associated with a sample having a quantity of organisms received in a container, the method comprising the steps of: acquiring, from a camera, an image the sample in the container; and determining a value of the variable of interest associated with the sample using the image.

### FIELD

This specification concerns systems and methods for evaluating a variable of interest concerning a sample having aquatic organisms and more particularly relates to such systems and methods which involve computer vision in evaluating the variable of interest.

### BACKGROUND

Aquaculture involves cultivating populations of aquatic organisms (e.g. fish) as they grow over time. Controlling the cultivated organisms and/or the amount of feed given to the cultivated organisms (sometimes in the form of smaller aquatic organisms), can be required to achieve satisfactory efficiency in terms of growth, survival rate and costs.

Published PCT application WO 2012/083461 describes methods and systems for estimating a relatively large quantity of organisms in a sample using the quantity of attenuation, by the sample, of a light signal. These methods and systems were satisfactory to a certain degree, but there always remains room for improvement. For instance, in order to provide a satisfactory degree of precision in the determination of the quantity of organisms it was known to perform a calibration beforehand to determine the quantity of attenuation associated to a known quantity of organisms. The quantity of organisms used in the calibration step was determined by hand-counting, which was time consuming.

### SUMMARY

One specific need occurs when it is desired to count a relatively large number of organisms. For example, fish egg producers typically counted the fish eggs one-by-one, in a time consuming process, in order to adequately evaluate the number of fish eggs which are present in a sample. In some cases, such as in the case of fish eggs for instance, these organisms can be amassed in a superposed manner to one another rather than being dispersed in a water medium during the step of counting. It was found that the volume which such amassed organisms occupy can be correlated to a quantity of organisms. A calibration process can be used beforehand, which can involve the determination of a (mean) unitary volume of organisms and a filling factor of the organisms (i.e. the amount of volume which is unused when the organisms are amassed, which depends on the shape and deformability of the organisms).

Accordingly, in accordance with an aspect, there is provided a method of determining a variable of interest associated with a sample of organisms received in a closed container having a contour wall extending upwardly from a closed bottom, the method comprising the steps of: receiving a given volume of the sample of organisms in the container such that the organisms are amassed to form a depth of organisms extending from the closed bottom to a given level of the contour wall; using a camera, acquiring an image of the sample of organisms received in the container; measuring an imaged level, in the image, corresponding to the given level of the contour wall to which the depth of organisms extends; and determining the quantity of organisms associated with the imaged level based on calibration data.

One other specific need is associated to the determination of a variable of interest concerning aquatic organisms of a sample, where the variable of interest can be correlated to an appearance of the aquatic organisms. For instance, the color of the organisms can be indicative of the health of the organisms (e.g. some bacterial, fungal and/or parasite diseases change the color of the organisms). In another example, the mean size and/or the size distribution of the organisms can be a variable of interest which is determined based on the appearance of the organisms, and more specifically by measuring the size of the organisms in a sample, for instance. There is thus a need for systems and methods which can be used to automate the determination of appearance-related variables of interest concerning the organisms in a sample.

In accordance with an aspect, there is provided a method of determining an appearance-related variable of interest associated with a sample of organisms received in a container, the method comprising the steps of: receiving a given volume of the sample of organisms in the container; using a camera at a fixed distance relative to the container, acquiring an image of the sample of organisms received in the container; and determining a value of the appearance-related variable of interest associated with the organisms using the image taken by the camera.

In accordance with another aspect, it was known to calibrate a system such as the one described in published PCT application WO 2012/083461 to subsequently allow satisfactory correlationship between an amount of received light (received light=emitted light−attenuation) and a quantity of organisms. More specifically, the calibration can include the determination of a biomass attenuation relationship (relationship indicative of amount of light absorbed by each individual organism), and can require the manual counting of a relatively large amount of organisms. There is thus a need to automate the determination of the biomass attenuation relationship associated with an organism, which, in turn, can help automating the calibration of the photometry system described in published PCT application WO 2012/083461.

In accordance with an aspect, there is provided a method of determining a biomass attenuation relationship associated with a sample of organisms received in a container, the method comprising the steps of: receiving a given volume of the sample of organisms in the container; using a camera, acquiring an image of the sample of organisms received in the container; determining a value of the quantity of organisms associated with the sample using the image; while a sample having the quantity of organisms previously determined is received in the container; emitting an initial intensity of diffused light onto the sample; the container receiving the diffused light and reflecting the diffused light through the sample, the sample thereby attenuating the initial intensity; measuring a reflected intensity of the diffused light; and comparing the reflected intensity to the quantity of organisms of the sample to obtain a biomass attenuation relation.

One need occurs when a large number of aquatic organisms need to be counted. For example, producers sometimes need to have a relatively good estimation of the number of aquatic organisms which are raised in order to provide the right amount of feed in order to increase growth and survival rate without wasting resources. Once the right amount of feed has been determined, the next challenge resides in actually providing the right amount of feed to the aquatic organisms, which can also require counting of organisms since the feed can be provided in the form of smaller living organisms (e.g. plankton). There is thus a need for systems and methods which can be used to automate the counting of the organisms in a sample. Accordingly, there are a very large number of organisms which remains to be counted for properly managing the production of organisms in the aquaculture industry.

In accordance with an aspect, there is provided a method of determining a variable of interest associated with a sample of organisms received in a container, the method comprising the steps of: receiving a given volume of the sample of organisms in the container; using a camera, acquiring an image of the sample received in the container; and determining a value of the variable of interest associated with the sample using the image.

The step of receiving the given volume can further comprise receiving the given volume of the sample of organisms in the container such that at least some of the organisms have a distinctive feature which do not overlap with the distinctive features of the other organisms, the method further comprising the step of: localizing the distinctive features of the organisms in the image; wherein said determining the value of the variable of interest associated with the volume received in the container is based on the localized distinctive features in the image.

The container can be a closed container having a contour wall extending upwardly from a closed bottom and known dimensions; wherein said receiving further comprises receiving the given volume of the sample in the container such that the organisms overlap with one another to form a layer of organisms extending upwardly from the closed bottom to a level of the contour wall, the method further comprising the steps of: obtaining a unitary volume associated with the organisms, the image comprising the sample and the interior of the container such that the image shows the level of the contour wall to which the layer of organisms extends, wherein the variable of interest is a volume of the layer of organisms; inferring the volume of the layer of organisms inside the container based on the level of the contour wall and the known dimensions of the container; and determining a quantity of organisms associated with the layer of organisms based on the unitary volume and the inferred volume of the layer of organisms.

The container can be an open container having an inlet, an outlet and a conduit between the inlet and the outlet, said receiving a volume of a sample of organisms further comprising receiving a flow of the sample of organisms at the inlet and flowing the volume of the sample across the conduit towards the outlet, the flow of the sample being such that at least some of the organisms have distinctive features which do not overlap with the distinctive features of the other organisms of the flow; localizing the distinctive features of the organisms in the image, the method further comprising the steps of determining a value of the variable of interest associated with the volume received in the container based on the localized distinctive features; and wherein said determining the value of the variable of interest associated with the volume received in the container is based on the localized distinctive features in the image.

In accordance with another aspect, there is provide a system for determining a value of a variable of interest concerning a sample of organisms, the system comprising: a container for receiving the sample; a structure mounted to the container and having a camera oriented towards the sample for acquiring an image of the sample received in the container; and a processor in communication with the camera, the processor being coupled with a computer-readable memory being configured for storing computer executable instructions that, when executed by the processor, perform the step of: determining a value of the variable of interest associated with the sample using the image.

In accordance with another aspect, there is provided a method of determining a variable of interest associated with a sample having a quantity of organisms received in a container, the method comprising the steps of: acquiring, from a camera, an image the sample in the container; and determining a value of the variable of interest associated with the sample using the image.

In accordance with another aspect, there is provided a method of determining a volume of a sample received in a container, the method comprising the steps of: receiving the sample in the container; acquiring, from a camera, an image of the sample received in the container; measuring an imaged level, in the image, corresponding to a level to which the sample extends in the container; determining a volume of the sample using the imaged level and calibration data.

Many further features and combinations thereof concerning the present improvements will appear to those skilled in the art following a reading of the instant disclosure.

### DESCRIPTION OF THE FIGURES

In the figures,

FIG. 1A is a cross-sectional, transversal view taken along a longitudinal axis of an example of a system for determining a variable of interest of a sample of fish, in accordance with an embodiment;

FIG. 1B is a cross-sectional, top view taken along section 1B-1B of the system shown in FIG. 1A, in accordance with an embodiment;

FIG. 1C is an example of an image acquired by the system shown in FIG. 1A, in accordance with an embodiment;

FIG. 2A is a cross-sectional, transversal view taken along a longitudinal axis of another example of a system for determining a variable of interest of a sample of fish eggs, in accordance with an embodiment;

FIG. 2B is a cross-sectional, top view taken along section 2B-2B of the system shown in FIG. 2A, in accordance with an embodiment;

FIG. 2C is an example of an image acquired by the system shown in FIG. 2A, in accordance with an embodiment;

FIG. 2D is a cross-sectional view taken along section 2D-2D of the system shown in FIG. 2A and showing a graduated contour wall, in accordance with an embodiment;

FIG. 2E is an example of a zoomed image acquired by the system and shows an enlarged portion of the image shown in FIG. 2C, in accordance with an embodiment;

FIG. 3A is a cross-sectional, transversal view taken along a longitudinal axis of another example of a system for determining a variable of interest having a camera and a light emitter, in accordance with an embodiment;

FIG. 3B is a cross-sectional, top view taken along section 3B-3B of the system shown in FIG. 3A, in accordance with an embodiment;

FIG. 3C is an example of an image acquired by the system shown in FIG. 3A, in accordance with an embodiment;

FIG. 4A is an oblique view of another example of a system for determining a value of a variable of interest, wherein the system has a camera and is mounted to an open container, in accordance with an embodiment;

FIG. 4B is an oblique view of another example of a system for determining a value of a variable of interest, wherein the system has a camera and a light emitter axially spaced along an open container, in accordance with an embodiment; and

FIG. 4C is an oblique view of another example of a system for determining a value of a variable of interest, wherein the system has a camera and a light emitter at a given axial position along an open container, in accordance with an embodiment.

These figures depict example embodiments for illustrative purposes, and variations, alternative configurations, alternative components and modifications may be made to these example embodiments.

### DETAILED DESCRIPTION

This disclosure describes methods and systems for determining a value of a variable of interest concerning a sample of aquatic organisms. Depending on the circumstances and on the embodiment, the variable of interest can be a quantity, an estimated unitary volume, a biomass, an appearance-related variable of interest such as a color, pigmentation or a presence of a disease, a depth, a position, a volume, a length, a width, an area and other variables of interest used in the field of aquaculture. The aquatic organisms can be fish, fish eggs, plankton and the like, depending on the application. As will be understood, although specific embodiments are described, embodiments which are best suited for determining given variables of interest associated with given organisms will be apparent for the skilled reader.

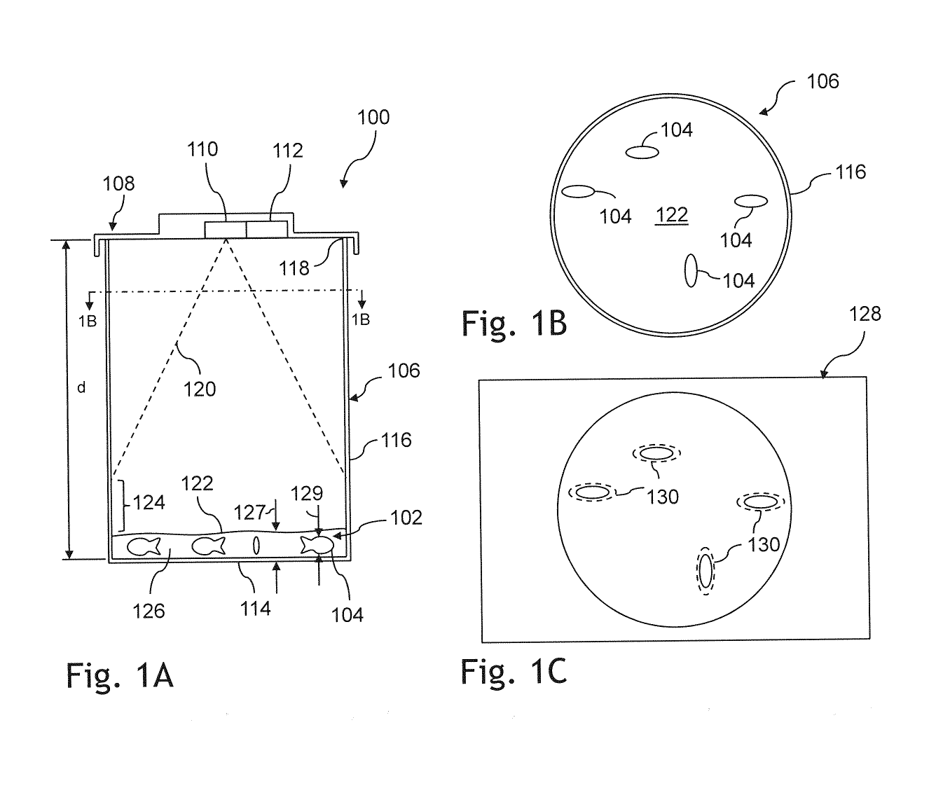

FIGS. 1A-B show an example of a system 100 for determining a value of a variable of interest, which is referred to simply as the “system”, concerning a sample 102 of organisms 104, in accordance with an embodiment. As shown, the system 100 has a container 106, a structure 108 mountable to the container 106 and one (or more) camera(s) 110 mounted to the structure in wired or wireless communication with a processing module 112. In the illustrated example, the container 106 can be referred to as a closed container due to its closed bottom 114 from which is extending a contour wall 116.

More specifically, the structure 108 is used to maintain the camera 110 at a given distance d from the closed bottom 114 of the container 106. As illustrated in FIG. 1A, the structure 108 is provided in the form of a lid, which is removably connected to an upper end 118 of the contour wall 116, opposite the closed bottom 114. In some embodiments, the container 106 and the structure 108 are made light hermetic to prevent light from entering the container 106 to avoid interference between ambient light and the system, for instance.

The camera 110 is so positioned that, when the container 106 receives the sample 102 of organisms 104, the camera 110 can image the sample 102, or a portion thereof, for further analysis by the system 100. In other words, the camera 110 has a field of view 120 which is oriented towards the sample 102. As depicted, the field of view 120 of the system 100 shown in FIG. 1A encompasses an entirety of the top surface 122 of the sample 102 as well as a portion 124 of the contour wall 116, as better seen in FIG. 10. In alternate embodiments, the field of view 120 can be limited to a given portion of the sample, as will be detailed further below.

Referring now to FIG. 1A, the processing module 112 typically has a processor, a memory and a power supply for powering the components of the system which require electrical power. In one embodiment, the power supply is a standalone power supply, such as a battery or a solar panel, to offer greater mobility to the system than by the use of a power cord, for instance. The memory can store readable instructions which, when executed by the processor, can perform steps for determining a value of a variable of interest based on the image.

In this embodiment, the processing module 112 is used to localize distinctive features of the organisms 104 in the image 128 and the value of the variable of interest is determined based on the localized distinctive features of the organisms 104. In order for the determination of the distinctive features of the organisms 104 to be satisfactory, the organisms 104 are dispersed in a layer of liquid medium 126 (e.g. water) or in no liquid medium.

As illustrated in the exemplary image 128 taken by the camera 110 of the system 100, the distinctive features are contours 130 of the organisms 104, but it is understood that the distinctive features can alternately be eyes, guts or any suitable imaged characteristic feature of the anatomy of the organisms 104 in question. Also, this embodiment can also be used with fish eggs or other suitable marine organisms.

In the illustrated embodiment shown in FIGS. 1A-B, the layer of liquid medium 126 has a depth 127 which is adjusted relatively to a dimension 129 of the organisms 104, the dimension 129 being substantially parallel to the depth 127. Such adjustment can help prevent or limit the amount of overlapping between the organisms 104 that can be seen by the camera 110, which can, in turn, help determining the value of the variable of interest with a satisfactory precision. In this specific embodiment, the dimension 129 of the organisms 104 can be referred to the thickness of the typical organism 104 as measured from the point of view of the camera 110. In other words, referring to the embodiment shown in FIGS. 1A-B, the dimension 129 is a height of the organisms 104. In an embodiment, the depth 127 is less than about two times the dimension 129 of the organisms 104, preferably less than about 1.5 times the dimension 129 of the organisms 104, and more preferably smaller than the dimension 129 of the organisms 104.

In the event where some organisms 104 of the sample 102 overlap, the system 100 can be specifically adapted to diagnose occurrences of overlapping and trigger an alarm and/or modify the value of the variable of interest. For instance, if the distinctive feature is the contour, the system 100 can be adapted to estimate the possible combinations of two overlapping contours and to localize such overlapping contours in the image 128. When overlapping contours are localized, the value of the variable of interest can be modified accordingly. Such modification can also apply for more than two overlapping organisms, depending on the circumstances.

It is understood that the image 128 can be digitally processed in order to enhance its contrast, for instance, to allow a more efficient localization of the distinctive features 130. For instance, the image 128 can be thresholded using a given intensity threshold such that any pixel of the image having an intensity lower than the given intensity threshold is set to black and that any pixel of the image having an intensity higher or equal to the given intensity threshold is set to white. In an alternate embodiment, the image 128 is also segmented in different portions such that the image is partitioned into multiple segments which help analyzing the image 128 by the system 100. As will be understood, other image processing techniques can be used.

It is contemplated that although only one image 128 is shown in FIG. 10, more than one image of the sample can also be used in order to determine the variable of interest. For instance, the images can be taken by a single camera or alternately by more than one camera. In the case where more than one camera is used, corresponding fields of view can differ which can allow avoiding the use of a more expensive zoom, for instance. In some embodiments, the images can be acquired sequentially in time in order to monitor the value of the variable of interest over time or to average the values to obtain an averaged value of the variable of interest. For instance, when analyzing small, mobile organisms individually with a zoomed-in lens in order to determine a feature such as length, taking a plurality of images while allowing the organisms to move between the images can allow achieving a greater degree of satisfaction in terms of statistical validity of the data once the length determined from the different individual pictures are averaged to determine a mean length, for instance.

In another embodiment, the system 100 can be used to determine another variable of interest such as a size, a depth, a length or a unitary volume of the organisms. In such an embodiment, the system 100 references the image 128 in space and factors in known dimensions of the container 106, a known field of view 120 of the camera 110 as well as a known distance d separating the camera 110 relative to the container 106 such that the size and/or volume of one organism 104 can be estimated using the image 128.

More specifically, the focal length of the camera 110 can be optimized for a specific distance between the camera 110 and the organisms 104 (e.g. the closed bottom 114) so that the sharpness of the image 128 may change as a function of the distance of the organism 104 relative to the camera 110. Accordingly, by quantifying the sharpness of the image, information about the depth of the organisms can be estimated.

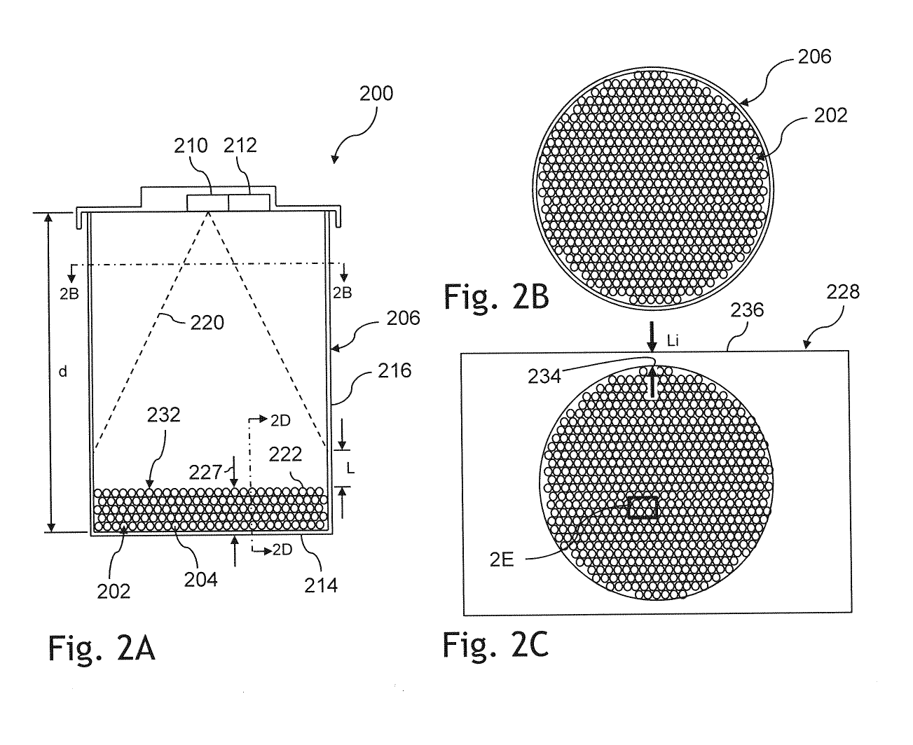

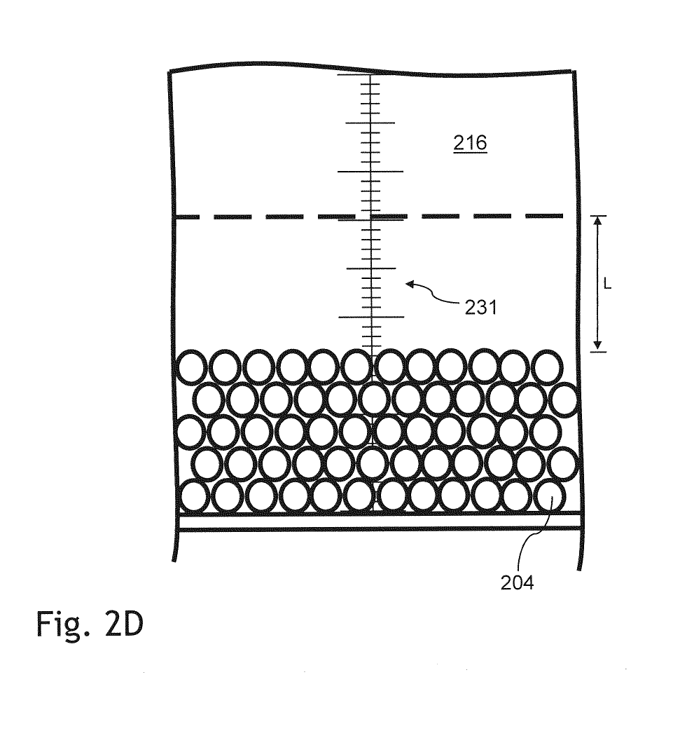

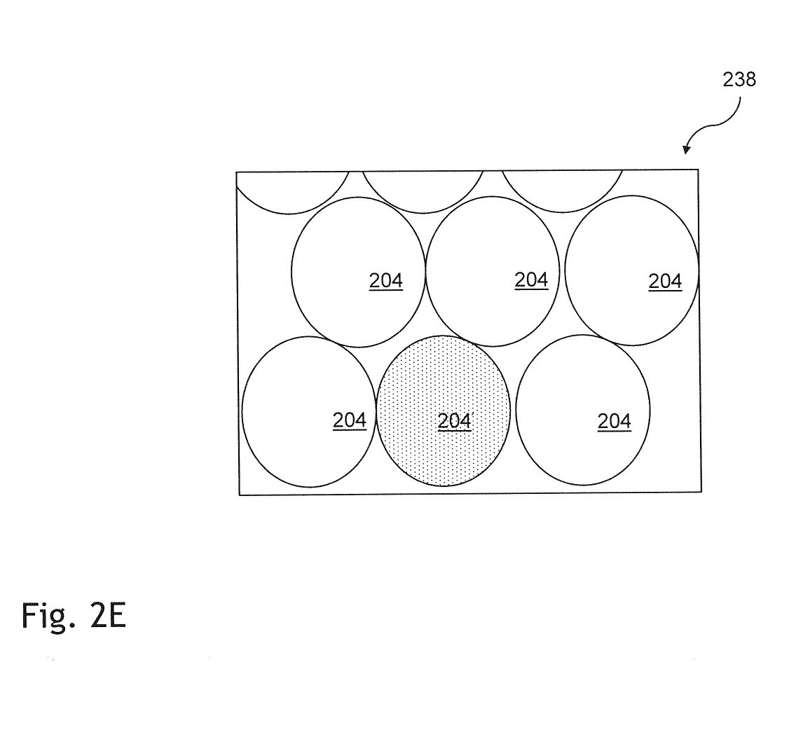

Now referring to FIGS. 2A-B, there is shown a system 200 for determining a value of a variable of interest, in accordance with an embodiment. In this specific embodiment, the value of the variable of interest, which is the quantity of organisms 204, is determined by correlating calibration data to computer vision. More specifically, in this example, the system 200 is used with a sample 202 of fish eggs exempt of liquid medium. The organisms 204 are received in the closed container 206 and are positioned such that the organisms 204 are amassed. The “amassed organisms” 204, which can be a plurality of organisms piled on top of each other and collected in a container under the effect of gravity, occupy a volume 232 of organisms 204 extending upwardly from the closed bottom 214 to a level L of the contour wall 216. The volume 232 occupied by the amassed organisms 204 may comprise a significant amount of empty space depending on the shape and compressibility/deformability of the organisms, or only a negligible amount of empty space. As will be detailed below, a filling factor can be used to determine the relationship between the number of organisms and the volume factoring in the amount of empty space if the amount of empty space is significant.

The system 200 is configured to measure the level L of the contour wall 216 which is reached by the volume 232 of organisms 204 using an image 228 such as the one shown in FIG. 2C. More specifically, the level L can be determined by measuring, in the image 228, an imaged level Li given by a number of pixels separating an imaged edge 234 of the depth of organisms and a reference point in the field of view 220 of the camera 210. The reference point can be an edge of the field of view 220, for instance, or another reference point. Once the imaged level Li is determined, it can be correlated to the quantity of organisms 204 of the sample using the calibration data.

In an embodiment, the calibration data associate the imaged level Li to the quantity of organisms 204. Such calibration data can be obtained by performing a calibration process which can include, for instance, placing a known quantity Nj of organisms 204 in the container, acquiring a calibration image and measuring a corresponding imaged level Li,j of the contour wall 216 using the calibration image. By repeating these steps at least another time, the calibration data, in this case provided in the form of a relationship Nj=f(Li,j), can be obtained for a given system 200 (e.g. for a given container 206). Accordingly, the system 200 can determine the quantity of organisms 204 in a sample 202 by correlating the imaged level Li, deemed proportional to the level L, to the quantity of organisms 204. The calibration data can also be provided in other suitable forms, as will be described herebelow.

In another embodiment, the system 200 is configured to determine the quantity of organisms 204 using calibration data which comprise known dimensions of the container 206 as well as a unitary volume associated with each organism 204. In this embodiment, the system 200 is configured to determine the imaged level Li in the image 228 and to infer a value of the volume 232 of the amassed organisms 204 based on the imaged level Li and on the known dimensions of the container 205. Once the value of the volume 232 of the amassed organisms 204 is inferred, the system 200 can determine the quantity of organisms 204 by correlating the unitary volume of the calibration data to the value of the volume of amassed organisms 204 inferred from the image 228.

It is noted that the calibration data used by the system 200 can include a filling factor, i.e. an estimated amount of emptiness between the organisms 204, associated with a given type of amassed organisms 204. For instance, fish eggs, which are typically substantially spherical, can have a different filling factor depending if they are amassed in a face-centered cubic (FCC) fashion or in a hexagonal close-packed (HCP) fashion. Accordingly, the system 200 can modify the quantity of organisms 204 based on the filling factor, which can typically bring the quantity of organisms 204 down by a certain extent. Depending on the organisms 204 and on their geometry, considering the filling factor in the determination of the quantity of organisms 204 can be negligible. In alternate embodiments, considering the filling factor of the organisms 204 is appropriate. In another embodiment, the emptiness between the organisms 204 can be filled with a liquid medium. In this specific embodiment, the filling factor can be modified when the amount of liquid medium is greater than the amount of emptiness that the sample would have if the sample had no liquid medium.

Further, the calibration data include a deformation factor associated with the organisms 204. In an embodiment, the deformation factor can cause the filling factor to vary as a function of the level L. For instance, when the deformation factor associated with the organisms 204 is significant, the filling factor of the organisms 204 closer to the closed bottom 214 can be higher than a filling factor of the organisms closer to the top surface 222. Accordingly, the quantity of organisms 204 determined by the system 200 can be modified by the deformation factor when it is believed that the deformability of the organisms 204 may influence the determination of the quantity of organisms 204 present in the sample 202. In another embodiment, when the deformation factor associated with the organisms 204 is low, the filling factor can be relatively constant throughout the sample 202.

In another embodiment, illustrated in FIG. 2D, the contour wall 216 has a graduated contour portion 231 to help converting the pixel distance Lp into the level L using the image 228.

In yet another embodiment, the systems 100 and 200 can be used to determine appearance-related variables of interest using the image(s) taken by the camera(s). The appearance-related variables of interest can include a color distribution, a pigmentation distribution, a size distribution, a presence of defect (e.g. parasites, diseases) and any useful information. Based on such appearance-related variables of interest, the systems 100 and 200 can associate a status to one or more organisms of the sample. For instance, the color of the organisms 204 can help determine if the organisms 204 are healthy or not (presence of bacterial-, fungal- and/or parasite-related diseases), which can be useful. Also, in another embodiment, it can be useful to determine the appearance-related variable of interest to the sample 202 of organisms 204 by either analyzing the sample 202 as a whole or by analyzing each individual organism 204 of the sample 202, for instance. Accordingly, it can also be useful to average the appearance-related variables of interest associated with the organisms 204 using more than one of the organisms present in a single image. In this embodiment, illumination of the sample for imaging purposes is maintained in order for the image to be comparable from one another. In case where the sample comprises a liquid medium, this embodiment can be used to determine a value of a variable of interest concerning the liquid medium.

In order to further determine the appearance-related variables of interest, the system 200 can have a field of view 220 which is limited to a given portion of the top surface of the sample 102. For instance, now referring to FIG. 2E, the field of view can be narrowed such that a zoomed image 238 is obtained. With such a zoomed image 238, the system 200 is configured to increase the resolution of the value of the variable of interest in embodiments where the variable of interest is the quantity of organisms, the estimated unitary volume of the organisms, and any appearance-related variable of interest associated with the organisms 204 and so forth, depending on the circumstances, notwithstanding the amount of overlapping between the organisms. Depending on the embodiment, the field of view can be set to a relatively small portion of the sample (e.g. 5%) in order to increase resolution. For instance, the zoomed image 238 of FIG. 2E shows several organisms 204 where the organism 204′ has a different color, which may be indicative that the organisms 204 has a particular disease, or a different degree of maturity.

Alternately, a similar method can be used to determine the volume of a liquid or semi-liquid sample (as opposed to a sample having amassed organisms with little or no liquid medium). Still alternately, a 3D image can be obtained using stereoscopic vision. A 3D image of the empty container can be used to determine the shape of the bottom of the container for calibration, and a 3D image of the container with the sample of organisms can be compared to the image of the empty container to determine the volume of the sample, which can in turn be associated to a quantity of organisms. In some embodiments, the camera can be very simple to reduce costs, and can be provided without zoom capability for instance.

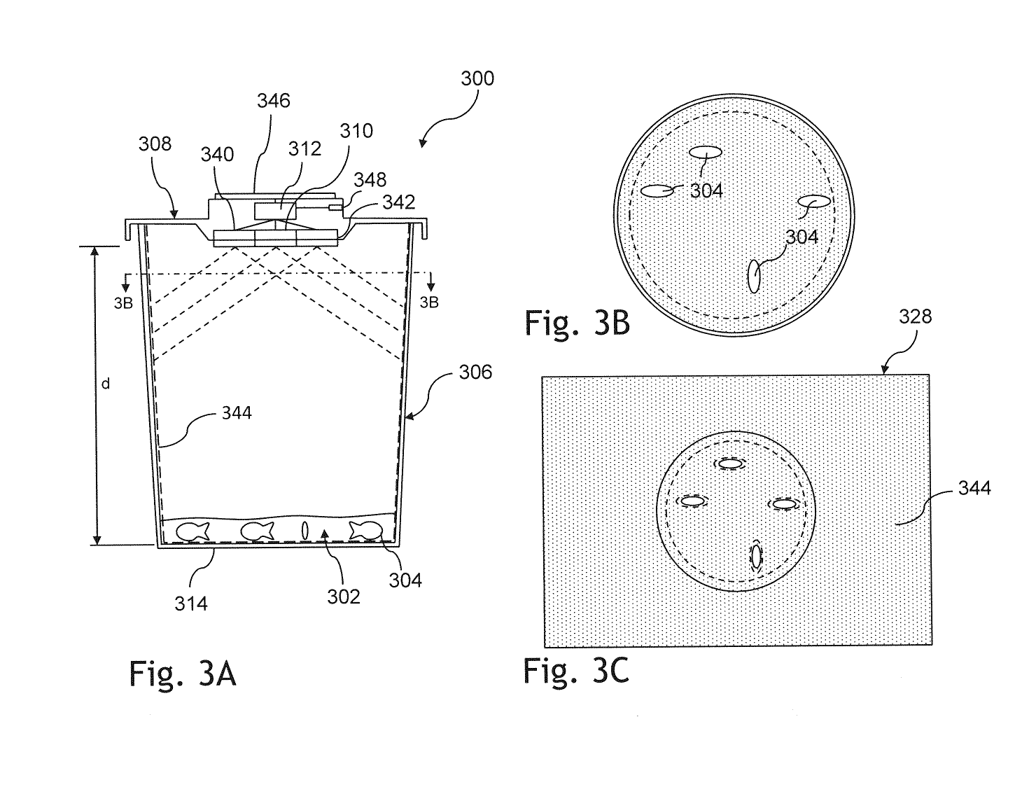

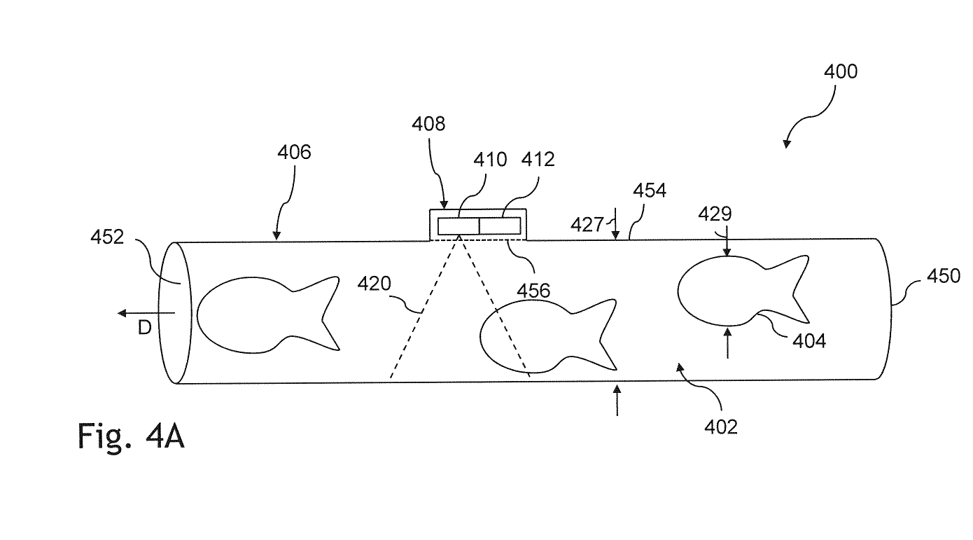

FIGS. 3A-B show another example of a system 300 for determining a value of a variable of interest of a sample 302 of organisms 304 using computer vision and photometry. In addition to the camera 310, the system 300 also has a light emitter 340 oriented towards the container 306 and towards the sample 302 and a light detector 342. In the illustrated embodiment, the light emitter 340 and the light detector 342 are mounted to the lid 308, both besides the camera 310, at the distance d from the closed bottom 314. In an alternate embodiment, rather than being a separate component, the camera can be used as the light detector 342, for instance.

When using a system such as the one described in published PCT application WO 2012/083461, determination of the value of the variable of interest requires a correlationship between an amount of received intensity of diffused light and a biomass attenuation relationship. Indeed, for a given system having a given reflective surface (e.g. white walls and bottom of a container), and where the organisms (biomass) of the sample are tested in a given liquid (e.g. water having a given attenuation factor) or tested alone (in a amassed relationship without liquid, e.g. eggs), the only remaining variable which affects the attenuation of the reflected light is the presence of the biomass, and so the attenuation relationship can be simplified to a biomass attenuation relationship. For ease of understanding, it is noted that the biomass attenuation relationship is typically a function which has units of “amount of energy absorbed per number of organism”. Accordingly, to determine the biomass attenuation relation, one had to measure the amount of energy which is absorbed by the organisms of a sample, divide this amount of energy by the total quantity (typically counted manually) of organisms present in that sample, and then repeat these steps for a given number of samples having differing quantity of organisms therein. Accordingly, using the quantity of organisms (determined using computer vision) and the energy absorbed by the organisms 304 (determined using photometry), the biomass attenuation relationship can be determined. In alternate embodiments, a more complex form of calibration can be performed to factor in other variables of a more general attenuation relationship, such as a calibration which factors in the attenuation of the walls and of a liquid medium for instance.

Still referring to FIGS. 3A-B, the system 300 combines computer vision to photometry so that determining the biomass attenuation relationship of the organisms 304 avoids hand counting the organisms 304. Therefore, the biomass attenuation coefficient can be obtained following a calibration process which includes the steps of receiving a sample of organisms 304 in the container 306, determining the value of the quantity of organisms 304 of the sample using computer vision, measuring an amount of energy absorbed by the organisms 304 inside the container 306, and dividing the amount of energy absorbed by the determined value of the quantity of organisms 304 of the sample. Typically, these steps of receiving, determining, measuring and dividing are performed more than one time in order to provide a biomass attenuation relationship having multiple biomass attenuation coefficients. In the specific case where the biomass attenuation relationship is directly proportional to the quantity of organisms 304, only one biomass attenuation coefficient (i.e. the slope m of the line y=m*x+b, wherein b=0) can suffice to determine the quantity of organisms of a given sample using the biomass attenuation coefficient.

In an embodiment, measuring the amount of energy absorbed by the organisms requires a referencing process in order to isolate the amount of energy which is actually absorbed by the organisms from the amount of energy that can be absorbed by the container, and/or by a liquid medium, if any. For each of the biomass attenuation coefficients of the biomass attenuation relation, the system 300 can be used to emit an initial intensity of diffused light towards a reference container exempt of organisms (for proper referencing, the reference container exempt of organisms can contain a liquid medium in cases where the sample comprises both organisms and a liquid medium) and to measure a reflected intensity of the diffused light which is reflected solely by the reference container (and by the liquid medium, if any). Then, the system 300 can be used to emit the initial intensity of diffused light towards the container 306 containing the sample 302 of organisms 304 and to measure a reflected intensity of the diffused light reflected by the container and by the sample. By comparing the initial intensity of diffused light, the intensity of diffused light reflected by the reference container and the reflected intensity of the diffused light reflected by the container and the sample, the amount of energy absorbed solely by the organisms 304 can be determined. In alternate embodiments, other suitable referencing processes can be used.

As mentioned above, having determined the biomass attenuation relationship associated with the organisms 304 using computer vision, the value of the quantity of organisms 304 can be provided using photometry. In alternate embodiments, it can be preferred to determine the biomass attenuation coefficient for a plurality of samples having different quantities of organisms 304 therein in order to obtain a statistically representative biomass attenuation relation. In another embodiment, once the biomass attenuation relationship is adequately determined using the combination of computer vision and photometry, the value of the variable of interest is determined using photometry since it is typically less power consuming than computer vision.

It is understood that for embodiments which use photometry, and possibly ones which use computer vision, the container 306 and/or the lid 308 are generally made of a material which is opaque for preventing external light from disturbing the lighting inside the container. In addition, the material is chosen to be reflective so that reflection of the diffused light onto the container 306 is increased. The container 306 and/or the lid 308 can thus be made of opaque white polymers. In the illustrated embodiment of FIG. 3A, the sample is received in a reflective liner 344 in order to increase the resolution of the system 300.

Also shown in FIG. 3A is that the system 300 has a user interface 346 on the lid 308. The user interface 346 is generally provided in the form of a touch screen which can be used to program the processing module 312 as well as displaying the value of the variable of interest, for instance. The system 300 also has communication ports and power outlets 348 for transferring data or power in and out the system 300.

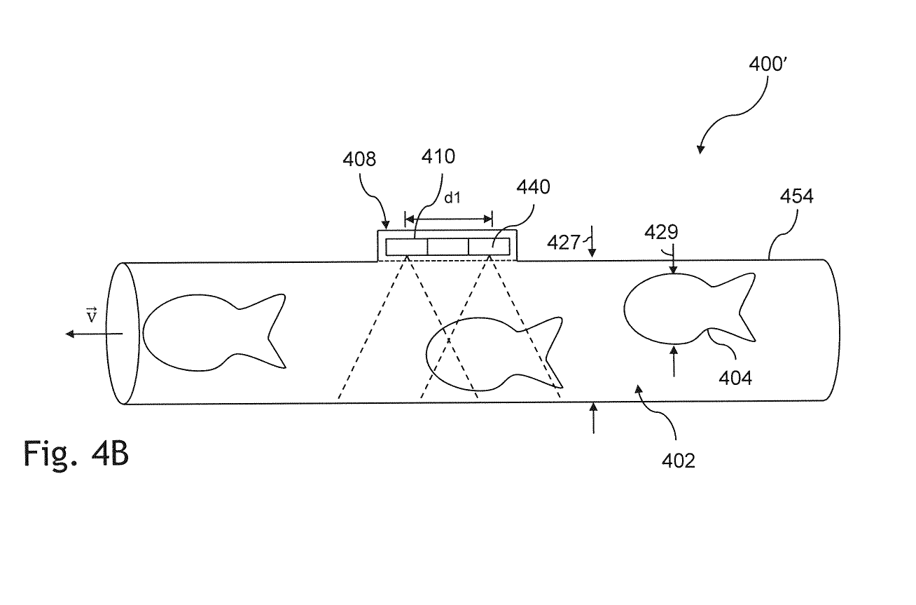

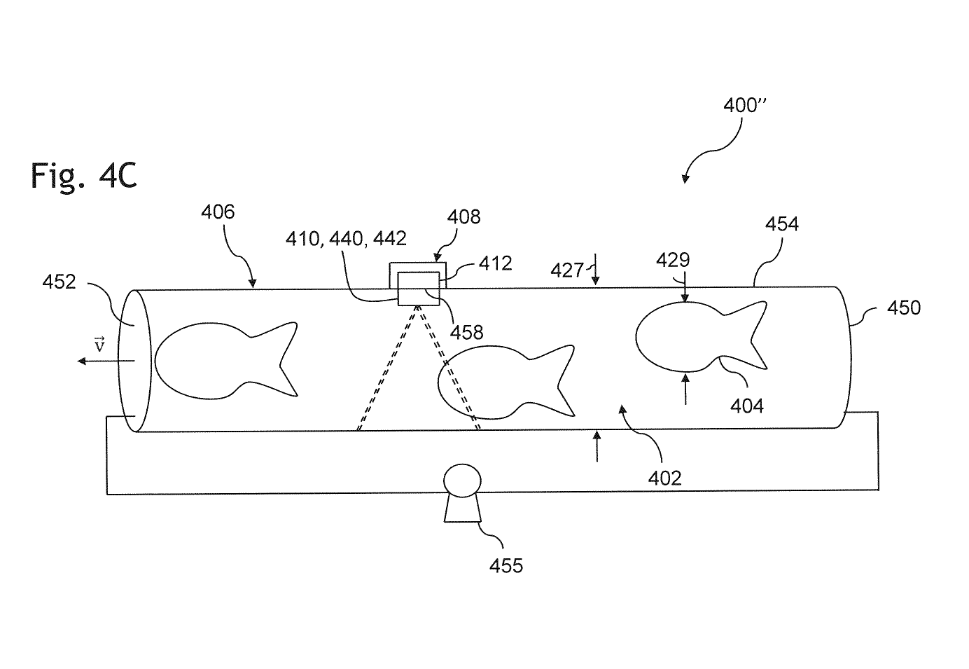

FIGS. 4A-C show different embodiments of a system 400 for determining a value of a variable of interest. In these embodiments, the container is provided in the form of an open container 406 having an inlet 450, an outlet 452 and a conduit 454 (e.g. a tubular conduit) therebetween. The sample of organisms 404 can thus be provided in a flow such that the organisms 404 flow along a given direction D from the inlet 450 towards the outlet 452 while the value of the variable of interest is determined.

In these embodiments, the flow has a depth 427 (relative to the point of view of the camera 410) chosen relatively to a dimension 429 associated with the organisms 404 in order to limit or prevent overlapping of the organisms 404 in the field of view 420. The dimension 429 is substantially parallel to the depth 427 such that, in the embodiments shown in FIGS. 4A-C, the dimension 429 corresponds to the height of the typically organism 404. It is noted that in alternate embodiments, the camera 410 can be positioned to image the organisms 404 along an axis perpendicular to the page of FIG. 4A, for instance, such that the dimension 429 of the organisms that is relevant is the thickness of the typical organism 404. In an embodiment, the depth 427 is less than about two times the dimension 429 of the organisms 404, preferably less than about 1.5 times the dimension 429 of the organisms 404, and more preferably smaller than the dimension 429 of the organisms 404.

Referring now to FIG. 4A, the system 400 has the camera 410 and the processing module 412 which are both housed in the structure 408 which is, in turn, hermetically secured to the conduit 454 via a transparent window 456 so that the field of view 420 of the camera 410 passes through the window 456 to image the organisms 404 as they flow. The camera 410 is positioned at an axial position along the conduit 454. For better accuracy, the amount of overlapping between the organisms 404 of the sample 402 is limited. In other words, the density of organisms of the volume which is imaged by the camera 410 is kept low.

FIG. 4B shows a system 400′ which can use computer vision as well as photometry. Indeed, the structure 408 has the camera 410 and the light emitter 440, mounted at two distinct axial positions along the conduit 454. Using the methods described above, the system 400′ can be used to determine a biomass attenuation relationship of the sample 402 of organisms 404. More specifically, in an embodiment, it is contemplated that the acquisition of the image and the acquisition of the reflected intensity are delayed, by a timer, from one another based on a spacing distance d1 (between the camera 410 and the light emitter 440) and on a linear speed V of the organisms along the conduit 454. Such timer thus help determining the value of the variable of interest of a given volume of the sample.

FIG. 4C shows a system 400″ which can also use computer vision and photometry. In this embodiment, the conduit 454 has an opening 458 by which the camera 410, the light emitter 440 and/or the light detector 442 hermetically project inside the conduit 454. In this embodiment, the camera 410, the emitter 440 and the light detector 442 are water-proof. As illustrated, the structure 408 hermetically maintains the components in the flow while keeping the processing module 412 dry. In an embodiment, the system 400″ can have a recirculating pump 455 which can be connected between the inlet 450 and the outlet 452 for recirculating the sample 402 of organisms 404 across the open container 406.

As can be understood, the examples described above and illustrated are intended to be exemplary only. For instance, the methods and systems described herein can be used for determining a value of a variable or interest concerning samples comprising crustaceans, molluscs and aquatic plants. In alternate embodiments, a combination of optical components such as lenses and filters can be used to optimize the resolution of the image as a function of the type of organisms interrogated. It is understood that the system can use different wavelengths of illumination, different types of suitable wavelength filters and different polarizing filters. Moreover, it is noted that the open container is not limited to be open solely to the inlet and to the outlet, it can also be open along the longitudinal axis of the open container (e.g. a river and the like). The expression ‘camera’ is used generally to refer to a device which can obtain and record visual images. In some embodiments, the camera can be a device having a digital camera chip have very simple optics and avoid the use of a zoom for instance, in other embodiments, the camera can include a zoom, whereas in still other embodiments, the camera can be provided in the form of a laser scanner, for instance. In most embodiments described above, the camera obtains a 2D image, but it will be noted that in alternate embodiments, the camera can obtain a 3D image. The scope is indicated by the appended claims.

1. A method of determining a variable of interest associated with a sample having a quantity of organisms received in a container, the method comprising the steps of:
acquiring, from a camera, an image the sample in the container; and
determining a value of the variable of interest associated with the sample using the image.

2. The method of claim 1, wherein the variable of interest is the quantity of organisms, wherein the container is a closed container having a contour wall extending upwardly from a closed bottom, and wherein said receiving further comprises receiving the sample in the container such that the organisms are amassed under the effect of gravity to form a depth of organisms extending from the closed bottom to a given level of the contour wall; the method further comprising the steps of:
measuring an imaged level, in the image, corresponding to the given level of the contour wall to which the depth of organisms extends; and
determining the quantity of organisms associated with the imaged level based on calibration data.

3. The method of claim 2, wherein said determining the imaged level further comprises determining the imaged level based on a number of pixels separating an imaged edge of the depth of organisms and a reference point in the image.

4. The method of claim 2, wherein the quantity of organisms determined is a second quantity of organisms, further comprising, prior to said determining the second quantity of organisms, providing the calibration data by performing the steps of:
acquiring a calibration image of a first quantity of organisms in the container;
measuring an imaged level, in the calibration image, corresponding to a level of the contour wall to which the determined quantity of organisms extends,
obtaining a value of the first quantity of organisms;
associating the value of the first quantity of organisms to the corresponding imaged level.

5. The method of claim 4 wherein the step of obtaining a value of the first quantity of organism includes:
acquiring, from a camera, an image of a sample having the first quantity of organisms in a container, the sample having a depth such that the organisms have a distinctive feature which do not overlap with the distinctive features of the other organisms;
localizing the distinctive features of the organisms in the image; and
determining the value of the first quantity of organisms associated with the sample based on counting of the localized distinctive features in the image.

6. The method of claim 5 wherein said providing the calibration data further comprises repeating acquiring, measuring, obtaining and associating with at least one other quantity of organisms.

7. The method of claim 2, wherein the container has known dimensions and wherein the calibration data comprises a unitary volume associated with the organisms, the method further comprising the steps of:
inferring a value of the volume of the depth of organisms inside the container based on the imaged level of the contour wall and the known dimensions of the container; and
determining the quantity of organisms associated with the value of the volume of the depth of organisms based on the unitary volume.

8. The method of claim 7, wherein the calibration data further comprises a filling factor associated with an amount of emptiness between the organisms, the method further comprising the step of:
modifying the determined quantity of organisms based on the filling factor.

9. The method of claim 8, wherein the amount of emptiness between organisms is partially filled with a liquid medium.

10. The method of claim 8, wherein the calibration data further comprises a deformation factor, the method further comprising:
determining the filling factor based on the deformation factor.

11. The method of claim 2, wherein the unitary volume of the calibration data associated with the organisms is estimated using the image of the sample.

12. The method of claim 2, wherein the organisms are fish eggs.

13. The method of claim 1, wherein the organisms have a distinctive feature which do not overlap with the distinctive features of the other organisms, the method further comprising the step of:
localizing the distinctive features of the organisms in the image;
wherein said determining the value of the variable of interest associated with the sample received in the container is based on the localized distinctive features in the image.

14. The method of claim 13, the sample has a depth adapted to avoid occurrences of distinctive features of the organisms overlapping with the distinctive features of the other organisms, further comprising adjusting the depth of the sample based on a dimension of the organisms, the dimension being substantially parallel to the depth.

15. The method of claim 14, wherein the depth is less than about two times the dimension of the organisms, preferably less than about 1.5 times the dimension of the organisms, and more preferably smaller than the dimension of the organisms.

16. The method of claim 13, wherein said determining further comprises modifying the value of the variable of interest based on an amount of overlapping organisms estimated using the image.

17. The method of claim 13, wherein the organisms are comprised in water.

18. The method of claim 13, wherein said localizing the distinctive features further comprises localizing at least one of a contour of the organism and an eye of the organism.

19. The method of claim 13, wherein said localizing further comprises a step of digitally processing the image, wherein said digitally processing includes one of modifying a contrast of the image, thresholding the image and segmenting of the image.

20. The method of claim 13, wherein said acquiring an image further comprises acquiring more than one image and wherein said determining further comprises determining more than one value of the variable of interest based on the more than one image.

21. The method of claim 20 further comprising averaging the values of the variable of interest to provide an averaged value of the variable of interest.

22. The method of claim 13, wherein said variable of interest is the quantity of organisms.

23. The method of claim 22, wherein the determined quantity of organisms is a first quantity of organisms, the method further comprising:
the sample having the first quantity of organisms attenuating an initial intensity of diffused light;
measuring the attenuated intensity of the diffused light; and
providing calibration data including associating the attenuated intensity of the diffused light to the determined quantity of organisms.

24. The method of claim 23 wherein said providing the calibration data further comprises repeating acquiring, measuring, obtaining and associating with at least one other quantity of organisms.

25. The method of claim 23, wherein said providing the calibration data includes a biomass attenuation relationship comprising biomass attenuation coefficients each associated to a corresponding attenuated intensity and to a corresponding quantity of organisms.

26. The method of claim 23, further comprising:
emitting the initial intensity of diffused light onto the sample, while the sample is in a container;
reflecting the initial intensity of diffused light against the container, across the sample,
receiving the attenuated intensity of the diffused light subsequently to said reflecting.

27. The method of claim 26, wherein said measuring the attenuated intensity of the diffused light comprises a referencing process comprising the steps of:
prior to said emitting, measuring and associating,
emitting the initial intensity of diffused light towards a reference container having no organisms therein; and
measuring a reference reflected intensity of the diffused light which is reflected by the reference container;
wherein said measuring a reflected intensity of the diffused light which is reflected by the organisms is based on the reference reflected intensity and on the initial intensity.

28. The method of claim 27, wherein the reference container has an amount of liquid medium similar to an amount of liquid medium of the sample, wherein the reference reflected intensity is reflected by the container and by the amount of liquid medium.

29. The method of claim 13, wherein said variable of interest is an estimated unitary volume of the organisms.

30. The method of claim 13, wherein the container is an open container having an inlet, an outlet and a conduit between the inlet and the outlet, said receiving the sample further comprising receiving a flow of the sample at the inlet and flowing the sample across the conduit towards the outlet, the flow of the sample having a depth such that at least some of the organisms have distinctive features which do not overlap with the distinctive features of the other organisms of the flow

31. The method of claim 30, wherein said acquiring an image further comprises acquiring more than one image taken at different axial positions along the conduit and delayed by a period of time based on the axial positions and on a linear speed of the flow; wherein said determined further comprises determining more than one value of the variable of interest based on the more than one image.

32. The method of claim 31 further comprising averaging the values of the variable of interest to provide an averaged value of the variable of interest.

33. The method of claim 1, wherein the variable of interest is an appearance-related variable of interest, wherein the camera is at a fixed distance relative to the container, the method further comprising the steps of:
determining a value of the appearance-related variable of interest associated with the organisms using the image taken by the camera.

34. The method of claim 33, wherein the appearance-related variable of interest is at least one of a presence of a defect, a size distribution and a color distribution of the organisms.

35. The method of claim 33, wherein said determining the value further comprises averaging the value of the appearance-related variable of interest associated with the organisms based on more than one of the organisms present in the image.

36. The method of claim 33, wherein said acquiring at least one image further comprises acquiring an image of a zoomed-in portion of the sample.

37-49. (canceled)
